(19) United States
(12) Patent Application Publication
(10) Pub. No.: US 2021/0158961 A1
(43) Pub. Date: May 27, 2021

(54) 1INTEGRATING ARTIFICIAL INTELLIGENCE BASED ANALYSES OF MEDICAL IMAGES INTO CLINICAL WORKFLOWS

(71) Applicant: Siemens Healthcare GmbH, Erlangen (DE)

(72) Inventors: Puneet Sharma, Princeton Junction, NJ (US); Dorin Comaniciu, Princeton Junction, NJ (US)

(21) Appl. No.: 16/690,251

(22) Filed: Nov. 21, 2019

(51) Int. Cl. G16H 50/20; G16H 30/40; G06N 5/04; G16H 15/00; G16H 10/60

(52) U.S. Cl. CPC G16H 50/20; G16H 30/40; G16H 10/60; G16H 15/00; G06N 5/04

(57) ABSTRACT

Systems and methods are provided for determining a recommended analysis of one or more input medical images using a trained machine learning network. The input medical images of a patient, artificial intelligence (AI) analysis information, user analysis information, and patient and input medical images information are received. The recommended analysis of the input medical images is determined using the trained machine learning network based on the AI analysis information, the user analysis information, and the patient and input medical images information. The recommended analysis of the input medical images is output.

### TECHNICAL FIELD

The present invention relates generally to integrating artificial intelligence (AI) based analyses of medical images into clinical workflows, and more particularly to determining a recommended analysis for evaluating medical images in a clinical workflow using a trained machine learning network.

### BACKGROUND

Artificial intelligence (AI) based algorithms have been applied to medical images for medical imaging analyses, such as, e.g., detection, segmentation, quantification, etc. With the advent of deep learning techniques and the availability of large amounts of training data, performance of such AI based algorithms has been progressively increasing in terms of diagnostic accuracy, sensitivity, and specificity. Nevertheless, the integration such AI based algorithms into clinical workflows remains a challenge. One of the primary issues is the lack of any standardized approach to determine, on a patient-specific basis, how the medical images are to be analyzed in the clinical workflow. In particular, there is currently no standardized approach to determine whether medical images are better suited for AI analysis, user analysis, or joint AI/user analysis.

### BRIEF SUMMARY OF THE INVENTION

In accordance with one or more embodiments, systems and methods are provided for determining a recommended analysis of one or more input medical images using a trained machine learning network. The input medical images of a patient, artificial intelligence (AI) analysis information, user analysis information, and patient and input medical images information are received. The recommended analysis of the input medical images is determined using the trained machine learning network based on the AI analysis information, the user analysis information, and the patient and input medical images information. The recommended analysis of the input medical images is output.

In one embodiment, the recommended analysis of the input medical images may be determined as one of the AI analysis, the user analysis, or a joint AI and user analysis of the input medical images. The AI analysis may be performed without performing the user analysis, and the user analysis may be performed without performing the AI analysis. The recommended analysis of the input medical images may also be determined as a score associated with each of the AI analysis, the user analysis, and the joint AI/user analysis.

In one embodiment, the information relating to the AI analysis of the input medical images may include one or more of inclusion and exclusion criterion of an AI algorithm of the AI analysis, performance metrics of the AI algorithm, a distribution of data from which the AI algorithm was trained, prior performance of the AI algorithm, specifications of the AI algorithm, and cost for using the AI algorithm. The information relating to the user analysis of the input medical images may include one or more of specialty of a user performing the user analysis, experience of the user, training of the user, certifications of the user, workload of the user, previous claims against the user, schedule and availability of the user, past performance of the user, reimbursements paid for image interpretation of the user, and response time of the user. The information relating to the patient or the input medical images may include one or more of a clinical indication triggering acquisition of the input medical images, characteristics of the patient, characteristics of an image acquisition device that acquired the input medical images, protocols for the acquisition of the input medical images, image quality of the input medical images, and a time of acquisition of the input medical images

These and other advantages of the invention will be apparent to those of ordinary skill in the art by reference to the following detailed description and the accompanying drawings.

### BRIEF DESCRIPTION OF THE DRAWINGS

FIG. 1 shows a high-level flow diagram for integrating artificial intelligence based analyses of medical images into a clinical workflow;

FIG. 2 shows a method for determining a recommended analysis of a medical image; and

FIG. 3 shows a high-level block diagram of a computer.

### DETAILED DESCRIPTION

The present invention generally relates to integrating artificial intelligence (AI) based analyses of medical images into clinical workflows. Embodiments of the present invention are described herein to give a visual understanding of methods for integrating AI based analyses of medical images into clinical workflows. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

Further, it should be understood that while embodiments of the present invention may be described with respect to integrating AI based analyses of medical images into a clinical workflow, the present invention is not so limited. Embodiment of the present invention may be applied for integrating analysis of any type of images into any type of workflow.

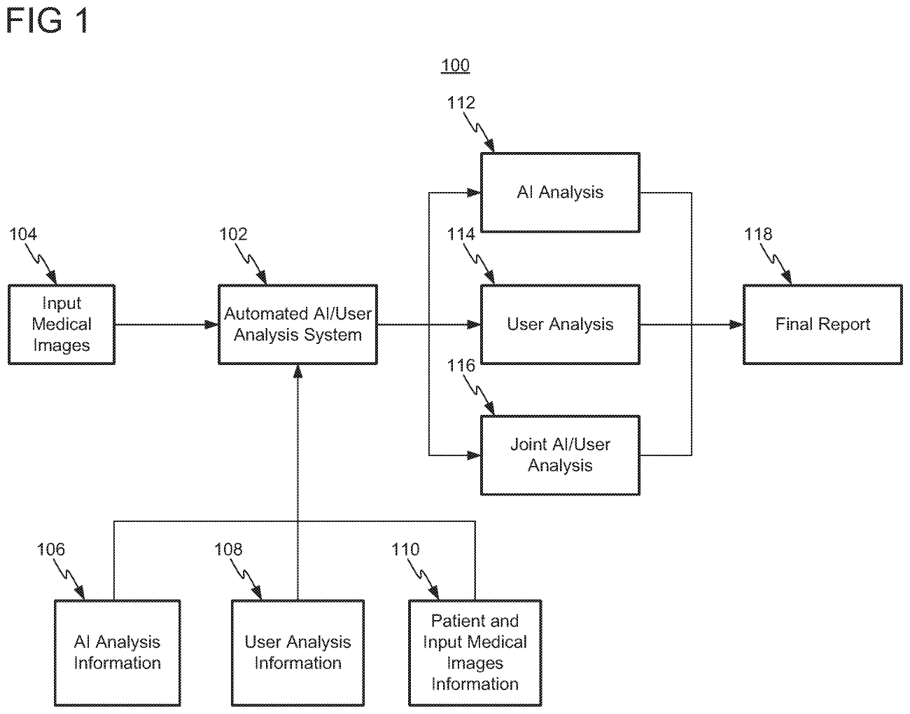

FIG. 1 shows a high-level flow diagram 100 for integrating AI based analyses of medical images into a clinical workflow, in accordance with one or more embodiments. Flow diagram 100 depicts automated AI/user analysis system 102 for determining a recommended analysis of one or more input medical images 104 of a patient to generate final report 118. Automated AI/user analysis system 102 determines the recommended analysis as, for example, one of an AI analysis 112, a user analysis 114, or a joint AI/user analysis 116 based on AI analysis information 106, user analysis information 108, and patient and input medical images information 110.

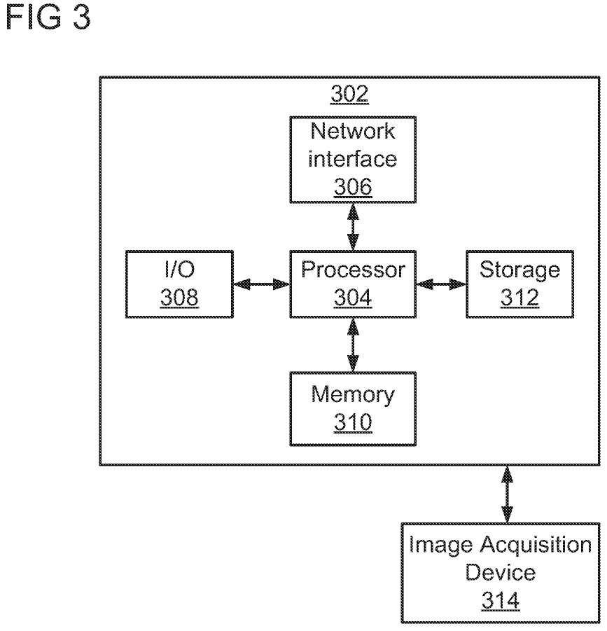

In one embodiment, automated AI/user analysis system 102 is implemented using a machine learning network executed using any suitable computing device, such as, e.g., computer 302 of FIG. 3. Automated AI/user analysis system 102 may be deployed on, or integrated with, an imaging scanner (e.g., image acquisition device 314 of FIG. 3), a picture archiving and communication system (PACS), a reading/post-processing application, or in a dedicated device that interfaces with the imaging scanner or PACS.

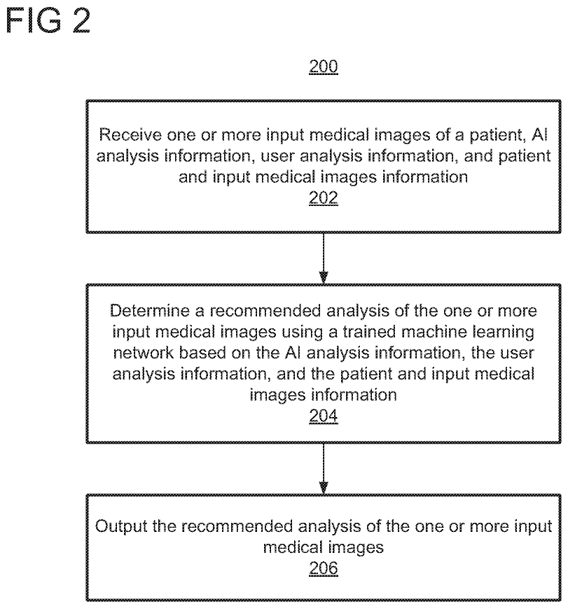

Advantageously, automated AI/user analysis system 102 determines how input medical images 104 are to be analyzed in a clinical workflow. Specifically, automated AI/user analysis system 102 determines which of the AI analysis 112, the user analysis 114, or the joint AI/user analysis 116 is better suited to evaluate the input medical images 104 to more efficiently integrate AI-based analyses into the clinical workflow (e.g., for radiological reading/reporting). Automated AI/user analysis system 102 thereby reduces operational costs while maintaining a high level of operational efficiency, diagnostic accuracy, and clinician satisfaction. Flow diagram 100 will be described in further detail with respect to method 200 of FIG. 2 below.

FIG. 2 shows a method 200 for determining a recommended analysis of a medical image, in accordance with one or more embodiments. Method 200 will be described with reference to flow diagram 100 of FIG. 1. In one embodiment, the steps of method 200 may be performed by a computing device, such as, e.g., automated AI/user analysis system 102 of FIG. 1.

At step 202, one or more input medical images 104 of a patient, AI analysis information 106, user analysis information 108, and patient and input medical images information 110 are received.

Input medical images 104 of the patient may be of any suitable modality, such as, e.g., computed tomography (CT), ultrasound (US), magnetic resonance imaging (MRI), etc. Input medical images 104 may be a single image of a region of interest of the patient, or a plurality of images of the region of interest of the patient (e.g., different views of the region of interest). Input medical images 104 may be received directly from an image acquisition device (e.g., image acquisition device 314 of FIG. 3) used to acquire the medical images. Alternatively, input medical images 104 may be received by loading previously acquired medical images from a storage or memory of a computer system (e.g., a PACS) or receiving medical images that have been transmitted from a remote computer system.

AI analysis information 106 includes any information relating to an AI analysis of input medical images 104. In one embodiment, AI analysis information 106 includes specifications of an AI algorithm for the AI analysis. For example, AI analysis information 106 may include inclusion and exclusion criterion of the AI algorithm, performance metrics (e.g., sensitivity, specificity, area under the receiver operating characteristic curve, operating point, etc.) of the AI algorithm, distribution of data from which the AI algorithm was trained (e.g., the original training and testing data on which the AI algorithm was developed), prior performance of the AI algorithm (e.g., in other clinical studies at one or more institutions), vendor specific specifications of the AI algorithm (e.g., runtime and/or turnaround time, network and computational resources required for executing the algorithm, etc.), and the effective cost-incurred by the institution for utilizing one “use” of the algorithm (taking into account the pricing model, e.g., pay per use, subscription, etc.).

User analysis information 108 includes any information relating to a user analysis of input medical images 104. For example, user analysis information 108 may include user (e.g., radiologist) specific data and task specific data. The user specific data may include, e.g., specialty or sub-specialty of a user (who may perform the user analysis), experience (e.g., years) of the user, training of the user, certifications of the user, workload of the user, previous claims (e.g., malpractice claims) against the user, schedule and availability of the user, etc. The task specific data may include, e.g., past performance (how the user performed on the task in terms of diagnostic accuracy, etc.) of the user for the task (e.g., the particular user analysis), reimbursements paid for the user performing the user analysis (e.g., the image interpretation), response time (i.e., turn-around time) for the task for the user, etc. In some embodiments, user analysis information 108 may include data on a plurality of users.

Patient and input medical images information 110 includes any information relating to the patient and/or input medical images 104. For example, patient and input medical images information 110 may include, e.g., the clinical indication triggering the acquisition of the input medical images, patient characteristics (e.g., clinical data, demographic data, or any other patient data in an electronic medical record), characteristics of the image acquisition device that acquired the input medical images, details of the acquisition protocol used to acquire the input medical images, image quality of the input medical images, time of the acquisition or reading of the input medical images (e.g., day time or night time), etc.

At step 204, a recommended analysis of the one or more input medical images 104 is determined using a trained machine learning network based on AI analysis information 106, user analysis information 108, and patient and input medical images information 110.

In one embodiment, the recommended analysis of input medical images 104 is one of an AI analysis 112, a user analysis 114, or a joint AI/user analysis 116. AI analysis 112 is any analysis of input medical images 104 using AI based algorithms, such as, e.g., AI based segmentation, detection, quantification, etc. User analysis 114 is any analysis of input medical images 104 performed by a user (e.g., radiologist). Joint AI/user analysis 116 is any analysis of input medical images 104 performed using both AI analysis and user analysis. In one example, joint AI/user analysis 116 may include first performing a user analysis on input medical images 104 and then performing an AI analysis to verify the user analysis. In another example, joint AI/user analysis 116 may include first performing an AI analysis on input medical images 104 and then performing a user analysis to verify the AI analysis. In one embodiment, AI analysis 112 refers to an AI only analysis of input medical images 104 performed without a user analysis and user analysis 114 refers to a user only analysis of input medical images 104 performed without an AI analysis. In one embodiment, user analysis information 108 includes information on a plurality of users (e.g., a staff radiologist and a tele-radiologist), and user analysis 114 and/or the joint AI/user analysis 116 may include an identification of a particular user for performing the user analysis.

In one embodiment, the recommended analysis of input medical images 104 is a score associated with each of AI analysis 112, user analysis 114, and joint AI/user analysis 116. The score may represent a probability that its associated analysis is best suited for analyzing input medical images 104.

The recommended analysis of input medical images 104 may be of any suitable granularity. For example, the recommended analysis may be for all input medical images 104 of an imaging study, a subset (e.g., a series or sequence) of input medical images 104, an individual image of input medical image 104, a region of interest within input medical image 104, etc. In one embodiment, the recommended analysis of input medical image 104 may comprise different analyses for different portions of input medical image 104. For example, the recommended analysis may comprise an AI-only analysis for a portion of a medical image and a user-only analysis for another portion of the medical image. Results of the AI-only analysis and the user-only analysis may then be combined to generate a final report of the analysis of input medical image 104.

The machine learning network may be any suitable machine learning based algorithm for determining a recommended analysis of input medical images 104. The machine learning network may be a supervised, unsupervised, or semi-supervised machine learning network. The machine learning network may be trained during a prior training or offline stage using training data and applied during an online or testing stage at step 204 to determine the recommended analysis of input medical images 104.

At step 206, the recommended analysis of input medical images 104 is output. For example, the recommended analysis can be output by displaying the recommended analysis on a display device of a computer system, storing the recommended analysis on a memory or storage of a computer system, or by transmitting the recommended analysis to a remote computer system. In response to the output of the recommended analysis, input medical images 104 may be analyzed according to the recommended analysis, e.g., by applying AI analysis 112, user analysis 114, or joint AI/user analysis 116 to input medical images 104 to generate final report 118 (e.g., a radiology report) interpreting input medical images 104.

It should be understood that the steps of method 200 may be repeatedly performed for each newly received input medical image to determine a recommended analysis for that newly received input medical image.

While the recommended analysis of input medical images 104 is described herein as being determined using a machine learning network, it should be understood that other implementations are also possible. In one embodiment, the recommended analysis of the input medical images is determined by formulating the determination of the recommended analysis as a multi-objective optimization problem having multiple hard and/or soft constraints. The multi-objective optimization problem may be solved using any suitable numerical optimization technique, such as, e.g., dynamic programming, evolutionary algorithms, etc. to determine an optimal or pareto optimal solution. The multi-objective optimization problem may account for any number of objectives, such as, e.g., minimizing overall cost while maintaining diagnostic accuracy above a particular threshold for a given period of time, maximizing diagnostic accuracy while maintaining cost below a particular threshold, etc.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 2. Certain steps or functions of the methods and workflows described herein, including one or more of the steps or functions of FIG. 2, may be performed by a server or by another processor in a network-based cloud-computing system. Certain steps or functions of the methods and workflows described herein, including one or more of the steps of FIG. 2, may be performed by a client computer in a network-based cloud computing system. The steps or functions of the methods and workflows described herein, including one or more of the steps of FIG. 2, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 2, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 302 that may be used to implement systems, apparatus, and methods described herein is depicted in FIG. 3. Computer 302 includes a processor 304 operatively coupled to a data storage device 312 and a memory 310. Processor 304 controls the overall operation of computer 302 by executing computer program instructions that define such operations. The computer program instructions may be stored in data storage device 312, or other computer readable medium, and loaded into memory 310 when execution of the computer program instructions is desired. Thus, the method and workflow steps or functions of FIG. 2 can be defined by the computer program instructions stored in memory 310 and/or data storage device 312 and controlled by processor 304 executing the computer program instructions. For example, the computer program instructions can be implemented as computer executable code programmed by one skilled in the art to perform the method and workflow steps or functions of FIG. 2. Accordingly, by executing the computer program instructions, the processor 304 executes the method and workflow steps or functions of FIG. 2. Computer 302 may also include one or more network interfaces 306 for communicating with other devices via a network. Computer 302 may also include one or more input/output devices 308 that enable user interaction with computer 302 (e.g., display, keyboard, mouse, speakers, buttons, etc.).

Processor 304 may include both general and special purpose microprocessors, and may be the sole processor or one of multiple processors of computer 302. Processor 304 may include one or more central processing units (CPUs), for example. Processor 304, data storage device 312, and/or memory 310 may include, be supplemented by, or incorporated in, one or more application-specific integrated circuits (ASICs) and/or one or more field programmable gate arrays (FPGAs).

Data storage device 312 and memory 310 each include a tangible non-transitory computer readable storage medium. Data storage device 312, and memory 310, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 308 may include peripherals, such as a printer, scanner, display screen, etc. For example, input/output devices 308 may include a display device such as a cathode ray tube (CRT) or liquid crystal display (LCD) monitor for displaying information to the user, a keyboard, and a pointing device such as a mouse or a trackball by which the user can provide input to computer 302.

An image acquisition device 314 can be connected to the computer 302 to input image data (e.g., medical images) to the computer 302. It is possible to implement the image acquisition device 314 and the computer 302 as one device. It is also possible that the image acquisition device 314 and the computer 302 communicate wirelessly through a network. In a possible embodiment, the computer 302 can be located remotely with respect to the image acquisition device 314.

Any or all of the systems and apparatus discussed herein may be implemented using one or more computers such as computer 302.

One skilled in the art will recognize that an implementation of an actual computer or computer system may have other structures and may contain other components as well, and that FIG. 3 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A method comprising:
receiving one or more input medical images of a patient, artificial intelligence (AI) analysis information of the one or more input medical images, user analysis information of the one or more input medical images, and patient and input medical images information;
determining a recommended analysis of the one or more input medical images using a trained machine learning network based on the AI analysis information, the user analysis information, and the patient and input medical images information; and
outputting the recommended analysis of the one or more input medical images.

2. The method of claim 1, wherein determining a recommended analysis of the one or more input medical images comprises:
determining one of an AI analysis of the one or more input medical images, a user analysis of the one or more input medical images, or a joint AI/user analysis of the one or more input medical images as the recommended analysis.

3. The method of claim 2, wherein the AI analysis is performed without performing the user analysis and the user analysis is performed without performing the AI analysis.

4. The method of claim 1, wherein determining a recommended analysis of the one or more input medical images comprises:
determining a score associated with each of an AI analysis of the one or more input medical images, a user analysis of the one or more input medical images, and a joint AI/user analysis of the one or more input medical images.

5. The method of claim 1, wherein the AI analysis information comprises one or more of inclusion and exclusion criterion of an AI algorithm, performance metrics of the AI algorithm, a distribution of data from which the AI algorithm was trained, prior performance of the AI algorithm, specifications of the AI algorithm, and cost for using the AI algorithm.

6. The method of claim 1, wherein the user analysis information comprises one or more of specialty of a user performing a user analysis, experience of the user, training of the user, certifications of the user, workload of the user, previous claims against the user, schedule and availability of the user, past performance of the user, reimbursements paid for image interpretation of the user, and response time of the user.

7. The method of claim 1, wherein the patient and input medical images information comprises one or more of a clinical indication triggering acquisition of the one or more input medical images, characteristics of the patient, characteristics of an image acquisition device that acquired the one or more input medical images, protocols for the acquisition of the one or more input medical images, image quality of the one or more input medical images, and a time of acquisition of the one or more input medical images.

8. An apparatus comprising:
means for receiving one or more input medical images of a patient, artificial intelligence (AI) analysis information of the one or more input medical images, user analysis information of the one or more input medical images, and patient and input medical images information;
means for determining a recommended analysis of the one or more input medical images using a trained machine learning network based on the AI analysis information, the user analysis information, and the patient and input medical images information; and
means for outputting the recommended analysis of the one or more input medical images.

9. The apparatus of claim 8, wherein the means for determining a recommended analysis of the one or more input medical images comprises:
means for determining one of an AI analysis of the one or more input medical images, a user analysis of the one or more input medical images, or a joint AI/user analysis of the one or more input medical images as the recommended analysis.

10. The apparatus of claim 9, wherein the AI analysis is performed without performing the user analysis and the user analysis is performed without performing the AI analysis.

11. The apparatus of claim 8, wherein the means for determining a recommended analysis of the one or more input medical images comprises:
means for determining a score associated with each of an AI analysis of the one or more input medical images, a user analysis of the one or more input medical images, and a joint AI/user analysis of the one or more input medical images.

12. The apparatus of claim 8, wherein the AI analysis information comprises one or more of inclusion and exclusion criterion of an AI algorithm, performance metrics of the AI algorithm, a distribution of data from which the AI algorithm was trained, prior performance of the AI algorithm, specifications of the AI algorithm, and cost for using the AI algorithm.

13. The apparatus of claim 8, wherein the user analysis information comprises one or more of specialty of a user performing a user analysis, experience of the user, training of the user, certifications of the user, workload of the user, previous claims against the user, schedule and availability of the user, past performance of the user, reimbursements paid for image interpretation of the user, and response time of the user.

14. The apparatus of claim 8, wherein the patient and input medical images information comprises one or more of a clinical indication triggering acquisition of the one or more input medical images, characteristics of the patient, characteristics of an image acquisition device that acquired the one or more input medical images, protocols for the acquisition of the one or more input medical images, image quality of the one or more input medical images, and a time of acquisition of the one or more input medical images.

15. A non-transitory computer readable medium storing computer program instructions, the computer program instructions when executed by a processor cause the processor to perform operations comprising:
receiving one or more input medical images of a patient, artificial intelligence (AI) analysis information of the one or more input medical images, user analysis information of the one or more input medical images, and patient and input medical images information;
determining a recommended analysis of the one or more input medical images using a trained machine learning network based on the AI analysis information, the user analysis information, and the patient and input medical images information; and
outputting the recommended analysis of the one or more input medical images.

16. The non-transitory computer readable medium of claim 15, wherein determining a recommended analysis of the one or more input medical images comprises:
determining one of an AI analysis of the one or more input medical images, a user analysis of the one or more input medical images, or a joint AI/user analysis of the one or more input medical images as the recommended analysis.

17. The non-transitory computer readable medium of claim 15, wherein determining a recommended analysis of the one or more input medical images comprises:
determining a score associated with each of an AI analysis of the one or more input medical images, a user analysis of the one or more input medical images, and a joint AI/user analysis of the one or more input medical images.

18. The non-transitory computer readable medium of claim 15, wherein the AI analysis information comprises one or more of inclusion and exclusion criterion of an AI algorithm, performance metrics of the AI algorithm, a distribution of data from which the AI algorithm was trained, prior performance of the AI algorithm, specifications of the AI algorithm, and cost for using the AI algorithm.

19. The non-transitory computer readable medium of claim 15, wherein the user analysis information comprises one or more of specialty of a user performing a user analysis, experience of the user, training of the user, certifications of the user, workload of the user, previous claims against the user, schedule and availability of the user, past performance of the user, reimbursements paid for image interpretation of the user, and response time of the user.

20. The non-transitory computer readable medium of claim 15, wherein the patient and input medical images information comprises one or more of a clinical indication triggering acquisition of the one or more input medical images, characteristics of the patient, characteristics of an image acquisition device that acquired the one or more input medical images, protocols for the acquisition of the one or more input medical images, image quality of the one or more input medical images, and a time of acquisition of the one or more input medical images.
